(19) United States
(12) Patent Application Publication
(10) Pub. No.: US 2011/0144455 A1
(43) Pub. Date: Jun. 16, 2011

(54) SYSTEMS AND METHODS FOR MONITORING A SUBJECT AT REST

(75) Inventors: Steven J. Young, Los Gatos, CA (US); Marco Kenneth Della Torre, San Francisco, CA (US); Yuri Zhovnirovsky, Albany, CA (US); Richard V. Rifredi, Los Gatos, CA (US)

(73) Assignee: BAM LABS, INC., Los Gatos, CA (US)

(21) Appl. No.: 13/035,397

(22) Filed: Feb. 25, 2011

Related U.S. Application Data
(63) Continuation-in-part of application No. 11/849,051, filed on Aug. 31, 2007.
Application No. 11/849,051 is a continuation-in-part of application No. 12/349,167, filed on Jan. 6, 2009.
(60) Provisional application No. 61/406,262, filed on Oct. 25, 2010.

(51) Int. Cl. A61B 5/00

(52) U.S. Cl. 600/301

(57) ABSTRACT

Disclosed herein are methods and devices for monitoring a subject at rest. One such device comprises a sensing unit having a fluid-filled bladder configured to be placed under a substrate on which the subject lays and a sensor in fluid communication with the bladder. The sensor is configured to sense pressure variations within the bladder generated by a heart beat, respiration and body weight of the subject and to generate signals indicative of the pressure variations. A processor is configured to receive the signals and to determine and generate output indicative of the subject's heart beat and respiration and presence on the substrate. An external device is configured to display one or more of the output.

### CROSS REFERENCE TO RELATED APPLICATIONS

This application claims priority to U.S. Provisional Patent Application Ser. No. 61/406,262 filed on Oct. 25, 2010. The present application is a continuation-in-part of U.S. patent application Ser. No. 11/849,051 filed on Aug. 31, 2007 and claiming priority to U.S. Provisional Application Ser. No. 60/846,642 filed on Sep. 22, 2006, and is a continuation-in-part of U.S. patent application Ser. No. 12/349,167 filed on Jan. 6, 2009, all of which are incorporated herein by reference.

### FIELD OF THE INVENTION

The present invention pertains to monitoring systems and programs for monitoring a subject or subjects at rest, and methods of performing the same.

### BACKGROUND

Historically, monitoring vital signs of a person has required expensive equipment, such as an electrocardiogram (EKG) or a ballistocardiograph (BCG). In addition to being prohibitively expensive for many situations (e.g., home use), both EKGs and BCGs can be too cumbersome for use outside of medical facilities. EKGs, for example, typically necessitate attaching electrodes to the bodies of users, while BCGs rely on large, heavy, and unaesthetic force-measuring platforms that users lie on.

In more recent times, devices including piezoelectric films or arrays of sensors have been developed to measure heart and respiration rates. A user can lie on the device, and the film or sensors can generate a signal indicate of the user's heart rate and/or respiration rate. However, these devices can also be expensive.

### SUMMARY

Disclosed herein are devices for monitoring a subject at rest. One such device comprises a sensing unit having a fluid-filled bladder configured to be placed under a substrate on which the subject lays and a sensor in fluid communication with the bladder. The sensor is configured to sense pressure variations within the bladder generated by a heart beat, respiration and body weight of the subject and to generate signals indicative of the pressure variations. A processor is configured to receive the signals and to determine and generate output indicative of the subject's heart beat and respiration and presence on the substrate. An external device is configured to display one or more of the output and may communicate with a wireless device to display output indicative of the subject's heart rate, respiration rate and presence on the substrate.

Another embodiment of the device for monitoring a subject at rest can be used to monitor patients. This embodiment comprises a sensing unit having a fluid-filled bladder configured to be placed on a substrate and a sensor in fluid communication with the bladder for generating signals in response to pressure variations within the bladder. A processing unit can be configured to receive the signals from the sensor and to generate from the signals a heart rate, respiration rate and static pressure of the subject. Means for providing to a user analyses based on the subject's heart rate, respiration rate and static pressure is included, with the analyses being an indication of the subject in bed or out of bed. Means for providing an alert message to a caregiver can also be provided.

Another embodiment of a device disclosed herein can be used for preventing pressure ulcers in subjects. This embodiment can comprise a sensing unit having a fluid-filled bladder configured to be placed on a substrate and a sensor in fluid communication with the bladder for generating signals in response to pressure variations within the bladder, wherein the pressure variations are generated from a heart rate, respiration rate and body weight of the subject. A processing unit can be configured to receive the signals from the sensor and to determine from the signals a baseline heart rate, respiration rate and static pressure and to determine changes in strength of each of the heart rate, respiration rate and static pressure. Means for providing to a user analyses based on the changes is included, wherein the analyses includes an indication of a need for a user action to prevent a pressure ulcer on the subject.

Another embodiment of a device disclosed herein is a portable biosensing device for use by a subject at home comprising a bladder configured to be filled with a fluid and placed on a substrate, a sensor in fluid communication with the bladder and configured to generate signals in response to pressure variations within the bladder, wherein the pressure variations are generated from a heart rate, respiration rate and body weight of the subject resting on the fluid-filled bladder, a means for filling the bladder with fluid, a processor configured to receive the signals from the sensor and determine the subject's heart rate and respiration rate and a database for storing the heart rate and respiration rate data.

Also disclosed herein are methods of monitoring a subject at rest. One embodiment of a method disclosed herein monitors a subject by first sensing with a sensor incident pressure waves generated by the subject resting on a monitoring device, wherein the monitoring device comprises a fluid-filled bladder configured to be placed on a substrate with the sensor in fluid communication with the bladder. Signals are generated indicative of the incident pressure waves and sending the signals to a processor. A heart beat, respiration and static pressure of the subject are discriminated from analysis of the signals and an individual fingerprint is determined based on heart beat, respiration and static pressure for the subject. Whether the subject is in an assigned bed, not in the assigned bed, or in a different bed can be determined based on the presence or absence of the individual fingerprint and this can be communicated an external device.

Another method disclosed herein is an embodiment that can prevent pressure ulcers. This method comprises sensing with a sensor incident pressure waves generated by the subject resting on a monitoring device, wherein the monitoring device comprises a fluid-filled bladder configured to be placed on a substrate with the sensor in fluid communication with the bladder. Signals are generated indicative of the incident pressure waves and sending the signals to a processor and a baseline heart rate, respiration and static pressure of the subject is determined from the signals. An amount of time the subject is resting on the monitoring device is determined, and data indicating amount of movement and degree of movement of the subject while the subject is resting based on changes in static pressure of the bladder, changes in strength of heart beat and changes in strength of respiration are determined. If the subject requires an action by a user to prevent a pressure ulcer based on the data is determined and the determination is communicated to the user.

Other embodiments of methods of monitoring one or more subjects at rest are disclosed. Another method of monitoring at least one subject at rest, wherein each subject is associated with a touchless monitoring system assembly, comprises receiving at predetermined intervals data about the subject from the associated touchless monitoring system, generating content representing the subject and the subject's data and displaying the content on a display. The touchless monitoring system assembly can comprise a fluid bladder, a sensor in fluid communication with the fluid bladder, and a processor. The sensor can be configured to determine a pressure within the fluid bladder and to send a signal indicative of the pressure to the processor, which can be configured to convert the signal to data. Receiving data from the touchless monitoring system can comprise receiving at predetermined intervals the data from the processor. The data can be communicated to the user through a bedside device with visual or audio indicator or communicated to a caregiver through a wired or wireless communication channel to a mobile device or personal computer.

Also disclosed herein are embodiments of monitoring programs for monitoring at least one subject at rest on a touchless monitoring system assembly. One exemplary embodiment comprises a monitoring system program configured to receive data from the touchless monitoring system assembly and to generate content based on the data, a display configured to display the content and a user interface. The touchless monitoring system assembly can comprise a fluid bladder, a sensor in fluid communication with the fluid bladder and a processor. The sensor can be configured to determine a pressure within the fluid bladder and to send a signal indicative of the pressure to the processor, which is configured to convert the signal to data that is received by the monitoring system program.

As used herein, data can include, but is not limited to, heart rate, respiration rate, length of sleep, quality of sleep, position, presence or absence in bed, blood pressure, tossing and turning movements, rolling movements, limb movements, and weight.

### BRIEF DESCRIPTION OF THE DRAWINGS

The description herein makes reference to the accompanying drawings, wherein like reference numerals refer to like parts throughout the several views, and wherein:

FIG. 1 is a schematic view of a touchless monitoring system apparatus for monitoring a subject at rest;

FIG. 2 is a diagram of the touchless monitoring system of FIG. 1;

FIG. 3 is a method of monitoring a subject at rest;

FIG. 4 is another method of monitoring a subject at rest;

FIG. 5 is a schematic view of a portable touchless monitoring system apparatus for monitoring a subject at rest;

FIG. 6 is a schematic view of another embodiment of a touchless monitoring system apparatus for monitoring a subject at rest;

FIG. 7 is a diagram of the touchless monitoring system of FIG. 1 with a monitoring program;

FIG. 8 is a login page generated by a monitoring system program disclosed herein;

FIG. 9 is a home page display screen generated by a monitoring system program disclosed herein;

FIG. 10 is a dashboard display screen generated by a monitoring system program disclosed herein;

FIG. 11 is a live view display screen generated by a monitoring system program disclosed herein;

FIG. 12 is a trend display screen generated by a monitoring system program disclosed herein;

FIG. 13 is a settings display screen generated by a monitoring system program disclosed herein;

FIG. 14 is another embodiment of a home page display screen generated by a monitoring system program disclosed herein; and

FIG. 15 is another embodiment of a home page display screen generated by a monitoring system program disclosed herein.

### DETAILED DESCRIPTION

Disclosed herein are embodiments of a monitoring system for monitoring at least one parameter of a resting subject. The monitoring system is a touchless system, meaning it does not come in contact with the subject. Conventional monitoring systems have electrodes, pressure cuffs, and the like that are placed on the subject. The touchless system can be transported for use by the same subject in different locations. The monitoring system includes the touchless monitoring apparatus and a monitoring program that receives, saves, manipulates and displays various data as content for use by a monitoring user.

The system apparatus or assembly that can determine and provide data about a subject to the monitoring program includes a fluid bladder against which the subject rests. A pump is in fluid communication with the fluid bladder, and the pump is operable to increase a fluid pressure within the fluid bladder. A pressure sensor can be used to monitor a subject at rest on the bladder. The pressure sensor is in fluid communication with the fluid bladder, and the sensor is operative to determine a pressure within the fluid bladder. A processing unit is configured to determine at least one parameter of the subject based on the pressure within the fluid bladder, the data comprising at least one parameter. A data base can store historical data including the pressure within the fluid bladder as raw data and one or more parameters about the subject that comprise the data. A processor can determine a quality correlation between the pressure within the fluid bladder and the at least parameter based on the data and historical data. A software program displays the data in the desired form, enabling the monitoring user to see detailed information about the subject who is using the fluid bladder.

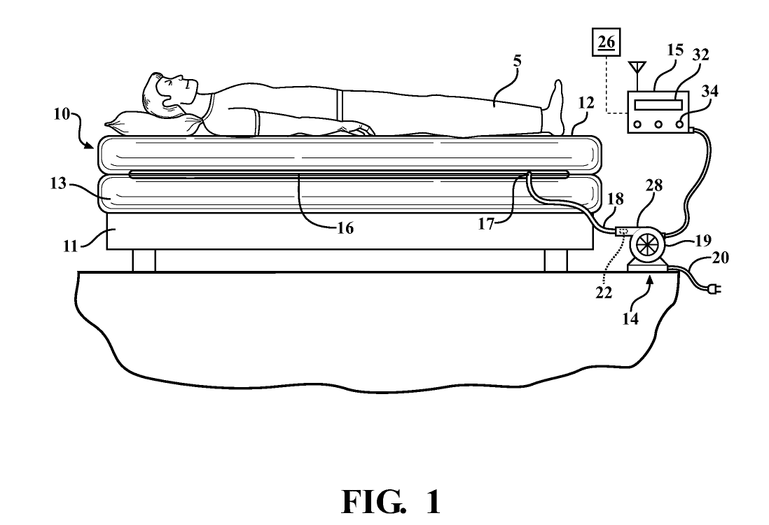

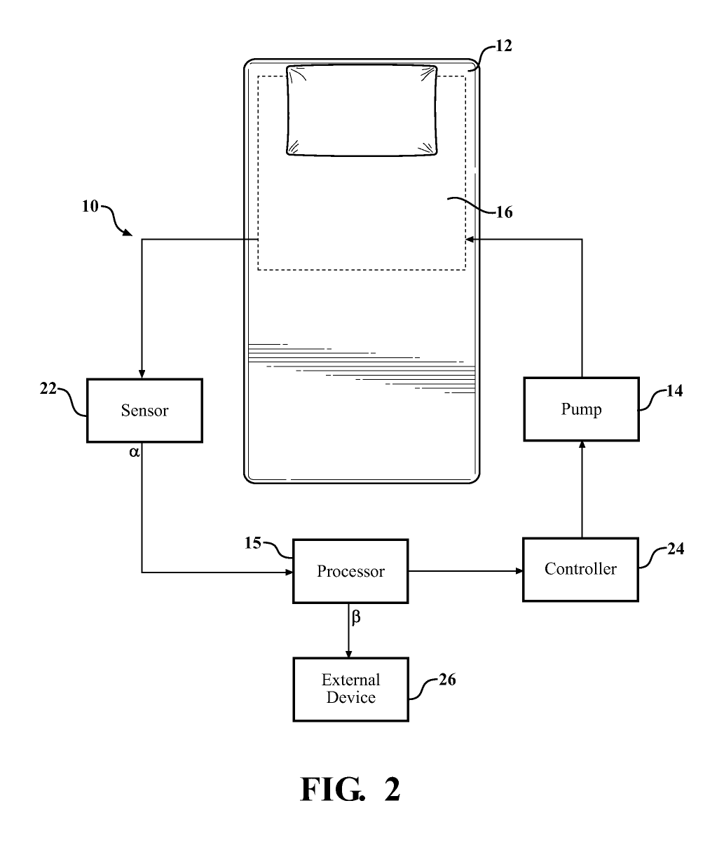

A monitoring system can be used to detect and measure one or more parameters of a subject 5, a subject being a person or an animal. For example, a monitoring system having both a monitoring apparatus 10 and an external monitor device 26 can be used with a bed frame 11 and mattress 12, with the apparatus 10 comprising a fluid bladder 16, a pump 14, and a processing unit 15 as shown in FIGS. 1 and 2. The system apparatus 10 can additionally include a padding layer 13 on top of and/or beneath the fluid bladder 16 as shown in FIG. 1. The padding layers 13 can include one or more of a foam pad, a box spring, an additional fluid bladder, a straw-filled pad, a feather-filled pad, a sawdust-filled pad, a spring-based pad, and/or another type of padding that offers flexibility and/or softness. Alternatively, the fluid bladder 16 can be sized for use in a chair, hospital bed, crib, or another structure that regularly supports an individual subject at rest.

The bladder 16 can hold air or another fluid, such as water. The fluid bladder 16 can be sized to have a surface area nearly as large as a surface area of a top side of the mattress 12, such as a king-size, queen-size, full, twin, or other sized mattress 12, to allow the detection of a user's vital signs regardless of the position of the user. Alternatively, the bladder 16 can have a smaller size so long as it is sufficient to cover a user's heart and/or lungs. For example, a size covering an area of the mattress 12 above which the user's heart and/or lungs are expected to be positioned (e.g., a one foot by one foot square for an adult user) can be used. Incident pressure waves caused by shifting body weight in response to cardiopulmonary activity induce a change in the measured pressure. The pressure in the fluid bladder 16 can vary depending on the amount of fluid in the bladder 16, whether a user is lying on the bladder 16, the heart rate of a user lying on the bladder 16, the respiration rate of a user lying on the bladder 16, and other movement of a user lying on the bladder 16 (e.g., rolling or limb movement).

The pump 14 can be a separate unit from the bladder 16 and can be fluidly coupled to an air inlet 17 of the bladder 16 via a hose 18 as shown in FIG. 1. The pump 14 can alternatively be integral with the bladder 16 such that the pump 14 can output high pressure fluid directly into the bladder 16 instead of through the hose 18. The pump 14 can be a rotary type pump or another type of pump. The pump 14 can include an electric line 20 for connection to a power source, or the pump 14 can include a self-contained power source, such as one or more batteries. The pump 14 can also include a data line for communication with the processing unit 15.

As shown in FIG. 1, the pump 14 can be packaged with a sensor 22. That is, the pump 14 and sensor 22 can be part of an integral unit. For example, a pump housing 19 that acts as a casing containing components of the pump 14 can also contain the sensor 22. The sensor 22 can be positioned within the pump housing 19 to detect an amount of air pressure in the hose 18. For example, the sensor 22 can be positioned in a portion of the pump 14 in communication with the hose 18, such as in fluid communication with the pressurized fluid outlet 28 of the pump 14 as shown in FIG. 1. Since the hose 18 can be in fluid communication with the bladder 16, the air pressure detected by the sensor 22 can indicate the air pressure in the bladder 16. While operation of the pump 14 may affect the pressure detected by the sensor 22, the pump 14 can operate only as required to maintain an average pressure within the bladder 16 (e.g., to replace any fluid that seeps out of the bladder 16).

Alternatively, the sensor 22 can also be located separate from the pump 14. The sensor 22 can be positioned such that the sensor 22 has a sensing side within the bladder 16 and a reference side outside of the bladder 16. In this case, the sensor 22 can be positioned in the seam of the bladder 16. The sensor 22 can include a semiconductor pressure sensor or another type of pressure sensor. Additionally, other types of sensors, such as a temperature sensor, can also be included. The sensor 22 can output a pressure signal a to the processing unit 15. The sensor 22 can be hard-wired to the processing unit 15, the sensor 22 can wirelessly communication with the processing unit 15 by way of a transmitter using, for example, a standard wireless protocol (e.g., IEEE 802.11, RF, Bluetooth, or 3G), or the sensor 22 can otherwise be coupled to the processing unit 15 for communication therewith.

A controller 24, which can be a microprocessor or another device, can receive a signal indicating the pressure of the bladder 16 from the processing unit 15 to control the pump 14 as shown in the flow diagram of FIG. 2 to operate the pump 14 to maintain or increase the pressure in the bladder 16. The controller 24 can also be in communication with an air release valve or other structure for releasing air from the bladder 16 such that the controller 24 can provide an instruction to decrease the fluid pressure in the bladder 16.

It is also contemplated herein that a self-inflating bladder can be used, thereby eliminating the need for the pump. Examples of self-inflating bladders that can be used with the embodiments herein include but are not limited to those disclosed in U.S. Pat. No. 6,651,277 to Marson and U.S. Pat. No. 4,624,877 to Lea et al., both incorporated in their entirety by reference.

The processing unit 15 can comprise one or more processors, digital signal processors, and different types of memory. Other peripheral devices may be used in addition to or in the place of the depicted hardware, which is not meant to imply limitations with respect to the embodiments. In addition to being able to be implemented on a variety of hardware platforms, the processing unit 15 can be a variety of software environments, such as various operating systems. The processing unit 15 maybe implemented in any electronic device, such as a desktop or laptop computer, a handheld or portable computer-like device and other electronic media players, cellular telephones, etc.

The processing unit 15 can analyze the pressure signal a and convert it to one or more parameters of the subject. These parameters, or data, include, but are not limited to, the following examples: heart rate, respiration rate, and changes in static pressure. These parameters can be analyzed to determine one or more of the following: length of sleep, quality of sleep, position, presence or absence in bed, blood pressure, tossing and turning movements, rolling movements, limb movements, weight and/or other data. More specifically, when a subject rests against the bladder 16, each of the subject's heart beats, breaths, and other movements can create a force on the bladder 16 that is transmitted to the sensor 22. As a result of the force input to the bladder 16 from the subject's movement, a wave will propagate through the bladder 16. The sensor 22 can detect the wave, transmitting the wave as a pressure signal a that can be computed into data such as a heart rate, respiratory rate, or other parameter regarding the subject.

The ability to detect heart beat and respiration, and changes in heart beat and respiration, in combination with changes in static pressure enables a user to gather detailed information about a subject. When only a change in pressure is detected, it is unknown from the signal alone what generated the change in pressure. For example, a suitcase may have been placed on the bladder. There is no way to verify that the signal is generated from the actual subject. The systems herein can develop a unique biological fingerprint for an individual with the combination of heart beat, respiration and weight, or static pressure. For example, one subject will generate a specific static pressure on the bladder due to the subject's weight. The subject also has a unique heart beat and respiration combination. With this data, the system can determine if that particular subject is resting on the bladder. In addition, the system herein can determine changes in position of the subject by the strength of each signal in combination with the other signals. For example, a subject is lying on his back if both of the heart beat and respiration signals are strong, and lying on his right side if the heart beat signal is weak while the respiration remains strong.

The processing unit 15 receives the signal a from the sensor 22 and can perform a pattern recognition algorithm or other calculation based on the amplified and filtered pressure signal a to determine the subject's heart rate, respiratory rate and derive other biosignal properties. For example, the algorithm or calculation can be based on assumptions that a heart rate portion of the signal a has a fundamental frequency in the range of 0.5-4.0 Hz and that a respiration rate portion of the signal a has a fundamental frequency in the range of less than 1 Hz. The processing unit 15 can also receive signals from other sensors (e.g., a temperature sensor). One processing unit 15 can be used to receive signals from a plurality of sensors in the same bladder, a plurality of sensors where some are inside and some are outside of the bladder, or a plurality of sensors from different bladders in use by different subjects.

The processing unit 15 can send status signals β indicating the parameters of the subject (e.g., heart rate and respiratory rate) to an external device 26 accessible to the user, such as a text messaging platform, data logger, printer, alarm system, alert siren, or other data acquisition or actuating device; or a computer (i.e., microprocessor) capable of performing analytical functions. These are non-limiting examples and other external devices 26 know to those skilled in the art can be used. The processing unit 15 can include a transmitter to relay the status signal β to the external device 26. The transmitter can be a wireless transmitter operating using a standard wireless protocol (e.g., IEEE 802.11, RF, Bluetooth, or 3G) or can alternatively be hardwired using a phone line, Ethernet line, or other connection.

Medical facilities such as hospitals, nursing homes and psychiatric institutions can use the monitoring system for many different reasons. A medical facility might use the monitoring system apparatus 10 in each bed, or each bed of a ward or floor, with each subject or patient associated with a system apparatus 10 or bed, providing instant information to the user of what patients are in bed and what beds are available to new patients. Because the system apparatus 10 is a non-contact monitoring system, patients need not be “hooked up” to the apparatus. The system does not need to be turned on by an employee or otherwise initiated. The system can begin to monitor the patient as soon as the patient rests against the bladder 16.

Medical facilities can use the system to continuously monitor vital signs of patients while the patient is in bed. The vital signs can be sent to an external device for monitoring by a doctor or nurse. However, there are many other uses for the apparatus 10 by medical facilities. Non-limiting examples are provided herein.

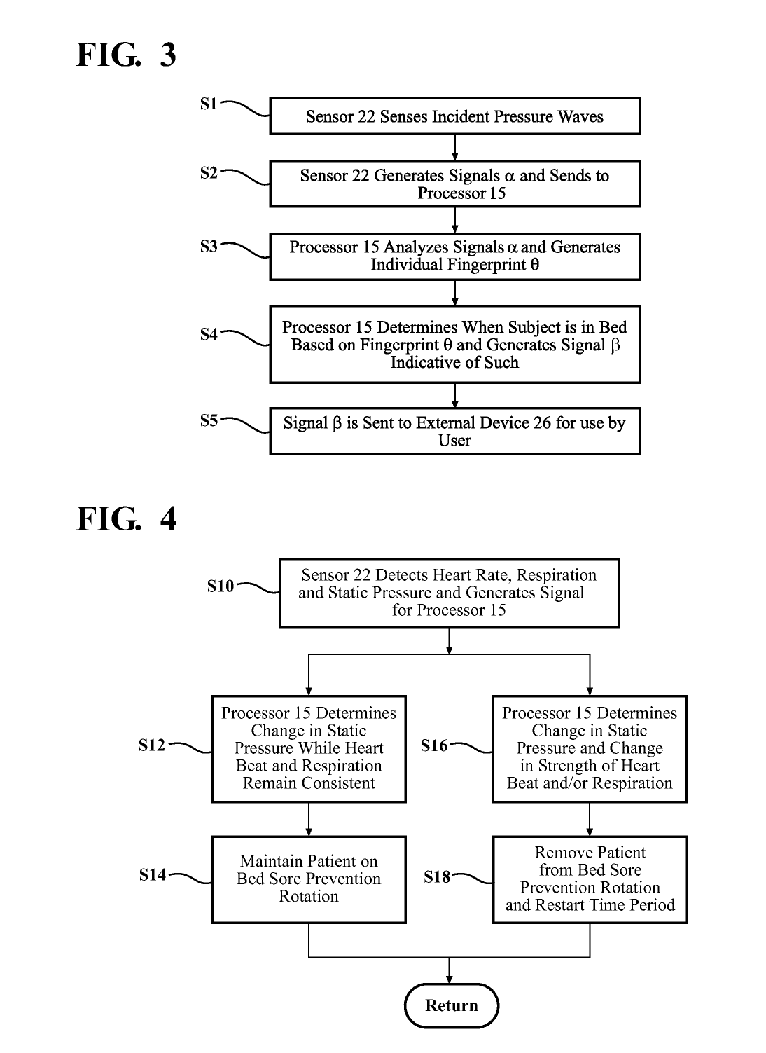

One embodiment of the monitoring system and method of use is for monitoring patients with dementia. A monitoring apparatus 10 as described is used for each patient being monitored. As illustrated in FIG. 3, the sensor 22 or sensors within the apparatus 10 sense incident pressure waves within the bladder in step S1 generated by the patient on the bladder 16. The sensor 22 generates signals α representing the pressure waves and sends to processor 15 in step S2. The processor 15 analyzes signals a and generates an individual fingerprint θ for the patient assigned to that particular monitoring apparatus 10, or bed in which that apparatus is used, in step S3. Once the fingerprint θ is developed, the processor 15 can determine when that particular person is in his or her bed. In step S4, the processor 15 generates a signal β indicative of whether or not the patient is in bed based on the patient's fingerprint θ. The signal β is then sent to the external device 26 accessible to the user, such as hospital or nursing home personnel, to monitor whether a patient with dementia is in bed or out of bed. Because of the individual biological fingerprint θ, the user can also determine if the patient is in someone else's bed. The system can be used to provide an alarm when a patient gets out of bed, alerting the staff or caregiver that the patient needs supervision.

For all patients, whether with dementia or not, an alert as to when a patient is out of bed can reduce the potential for falls, a problem medical facilities work to reduce. Supervision can be provided to a patient when the user receives an indication that a patient's heart rate and respiration are no longer sensed. This can be an indication that the patient has sat up and is preparing to get out of bed. Being able to provide assistance to patients as they get out of bed can reduce the number of slip and fall accidents in a medical facility setting.

The data generated by the processor 15 in the monitoring system described in FIG. 3 can also provide guidance for staffing requirements. This guidance may be particularly useful to determine nighttime staffing requirements. For example, in an institution or medical facility with long term care, the monitoring system can be used to track the number of times each patient gets out of bed by tracking the combination of static pressure, heart rate and/or respiration. The length of time the patient stays out of bed, as well as the overall length of time the patient sleeps, can also be tracked with the same data. This data can be stored and used to ascertain the average activity of the patients during the nighttime. The activity of the patients during the nighttime hours can determine the amount of staffing required to sufficiently provide for the safety and well being of the patients.

The monitoring system can also be used to monitor a patient's time in bed and movement in bed. Another embodiment of the monitoring system and method of use is for the prevention of bed sores, also called pressure ulcers. Conventional prevention of bed sores is attained by moving patients based on a schedule of time periods. Some methods sense pressure changes in a pad or plate. When only pressure is monitored, it is difficult to distinguish the extent of movement of the subject. For example, a light weight subject may completely turn over, generating incident waves of strength A. A subject with more weight may only move a limb and generate incident waves of the same strength A. The monitoring system as disclosed can detect heart rate and respiration, as well as static pressure changes in the bladder 16. When a subject simply moves a limb, the strength of the heart beat and respiration signal do not change, while the static pressure does change. However, when a subject rolls over onto his side, the strength of at least one of the heart beat and respiration decreases, along with the change in static pressure. Because heart rate and respiration are still detected, it is clear the subject is still in bed. However, the change in the strength of the heart beat and/or respiration indicates that the subject has changed position enough such that he does not need to be rolled over for bed sore prevention.

As shown in FIG. 4, the sensor 22 detects heart rate, respiration and static pressure and generates a signal α that is sent to the processor 15 in step S10. The processor 15 analyzes the signals sent by the sensor 22. If the processor 15 determines that there has been a change in static pressure while heart beat and respiration remain consistent, as in Step 12, the patient has moved, but not sufficiently to prevent a pressure ulcer. Accordingly, the patient remains on the list of those requiring turning over by staff, as in step S14. If the processor 15 determines that there has been a change in static pressure and a change in the strength of the heart beat and/or respiration, as in Step 16, the patient has turned over sufficiently to prevent a pressure ulcer. Accordingly, the patient is noted as not requiring turning over by the staff. The time period within which a patient is turned over is restarted.

Medical facilities and sleep clinics can monitor patients sleep without the need for electrodes contacting the patient, for use in determining a subject's sleep quotient and particular sleeping patterns of individual patients. For example, sleep apnea or restless leg syndrome can be diagnosed with the system. Sleep apnea can be diagnosed using heart rate, respiration and static pressure. Sleep apnea in a subject can be indicated by a change in static pressure and heart rate while respiration ceases. Restless leg syndrome can be diagnosed by frequent changes in static pressure without a change in heart rate and respiration.

The unique biological fingerprint θ associated with an individual can be used to monitor the compliance of regulations by, for example, prison release programs and half way houses. These programs have regulations to which participants must comply in order to remain in the program. As non-limiting examples, participants may be required to be in bed from 11 pm to 7 am, and participants may be required to refrain from drug use. Because the system is a non-contact monitoring system with the bladder of the system located under a mattress, there is no set up when the subject lies down or rests against the bladder. If desired, the subject need not know the monitoring system is there. However, due to the unique biological fingerprint θ that can be generated for each participant, the system cannot easily be manipulated to produce false data.

Current state fiscal crises and significant overcrowding at criminal justice facilities around the country have placed an intense focus on both community corrections and re-entry initiatives at both the state and federal levels. Such facilities to which prisoners are released can equip each bed with a monitoring apparatus 10 as described herein. Each apparatus 10 can communicate with a single processor 15 or more than one processor. The processor 15 can generate data to one or more external devices 26 that can be actively monitored by one or more users. In addition or in the alternative, the data can be saved for review at a later date. The processor 15 can determine the time in which the subject is in bed by the time the unique fingerprint θ is detected. Whether the subject remained in bed all night can be documented. Potential stress, alcohol or drug use can be detected by a change in heart beat and/or respiration. The ability to monitor compliance with a curfew, for example, without a device such as an ankle tether, can improve morale, decrease data manipulation, and reduce staffing costs.

The monitoring system can be used for many applications in a home setting. Because the monitoring apparatus 10 is a touchless system and requires little installation, there is no extensive training, preparation, or change in a subject's behavior in order to incorporate the system into regular use. A medical facility might send system apparatus 10 home with a patient upon discharge and use an on-site monitoring program described below to actively monitor that patient's parameters for a period of time following major surgery, for example. Professional home health care providers can use the system to enhance their capabilities and improve care. Non-professional care givers can use the monitoring system to gather data for physicians, set reminders for the turning of a patient or providing medication, etc. Periodic updates can be wirelessly sent to a medical professional. Professional home health care workers and non-professional caregivers can utilize the monitoring system for family members with dementia as described above.

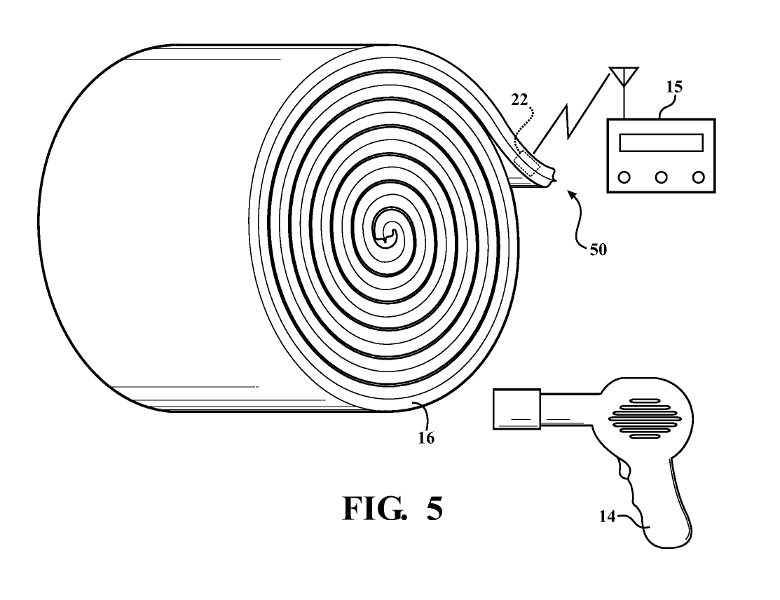

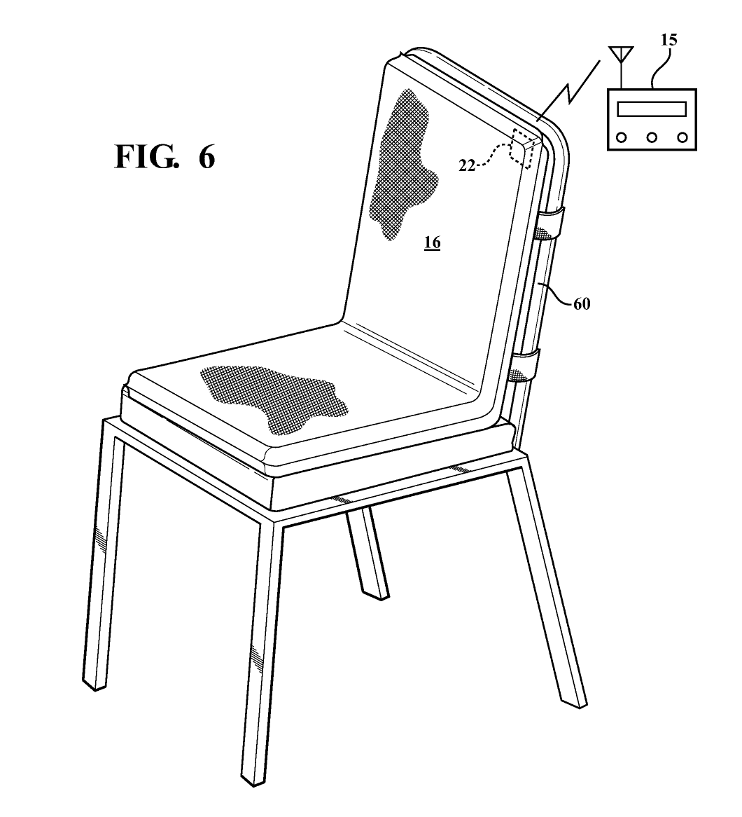

Another embodiment of the monitoring system disclosed herein is a portable monitoring device 50 for discharged patients. Patients discharged after post cardiac trauma or surgery, for example, can be provided the portable monitoring device 50 to take home with them. The portable device 50 shown in FIG. 5 includes the bladder 16, which can roll up into a portable size, the pump 14, the sensor 22, and the processor 15. The discharged patient can unfold the bladder 16 and place it under a mattress. The pump 14 can be an electrical pump that simply requires plugging in to operate to fill up the bladder 16 to the required pressure with fluid, such as air. The sensor 22 can be already permanently provided in the bladder 16, so that the discharged patient need not do anything with the sensor 22. The processor 15 can be a small unit that can just be placed on a night stand or the floor. The processor 15 can communicate with an external device 26 wirelessly, or a USB device can be used to collect data which is later brought by the patient or a caregiver for downloading by medical personal for reviewing. If the data is transmitted wirelessly, the physician has the benefit of accessing real time vital statistics of the discharged patient. The stored data can be trended over time as desired.

An individual can utilize an embodiment of the monitoring system for many reasons, such as to monitor sleep patterns, monitor overall health, monitor health during physical training, and monitor health during dieting. The data gathered by the individual can alert him or his doctor of sleep issues based on long-term sleep trends, such as sleep apnea or restless leg syndrome. The data can provide other health information such as the subject's fitness level, cardiovascular condition, etc. The data can simply be stored in a data base for use in the future in the event a medical condition or event occurs.

An individual can use the portable monitoring device 50 or an apparatus 10 for use in his or her home for at least the reasons discussed. If the individual is monitoring his or her sleep patterns, the user would set up the fluid bladder 16 of the apparatus 10 in his bed under the mattress 12 as described above. If the individual is monitoring his vital signs only, the individual may choose to position the bladder 16 of the apparatus 10 in a chair 60 as shown in FIG. 6 rather than a bed so that the system monitors the user's vital signs when he is sitting. The individual may, for example, work out and periodically sit in the chair against the bladder 16 to get a reading of his heart rate or respiration at desired intervals. The bladder 16 can be positioned on both the seat of the chair and the back of the chair to better read both weight and heart beat and respiration. It is also contemplated that the bladder 16 can only be located on the back of the chair 60 to monitor heart rate and respiration of the subject.

A parent or caregiver can use the monitoring system to monitor a baby's vital signs and movement while the baby is in a crib. The bladder 16 of the apparatus 10 can be placed under the crib mattress. An alarm can sound, for example, when the infant's breathing has stopped, alerting the parents to the need for intervention. A parent or caregiver may also use the monitoring system on children, perhaps through a physician, to determine if the child is getting sufficient sleep or if a lack of sleep is contributing to other behavioral manifestations.

The applications described are not meant to be limiting. Other applications consistent with the scope of the disclosure are contemplated. Each of the applications can be used with an external monitor device 26 as noted. One such external device 26 can be a monitoring program 100 as described herein.

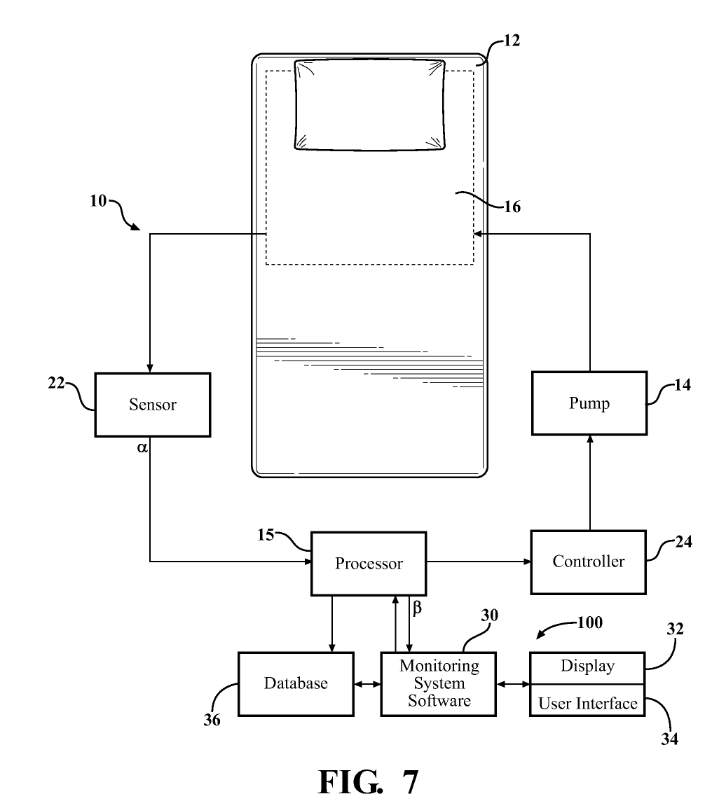

The monitoring program 100 is shown in FIG. 7 with the monitoring apparatus 10. The monitoring program 100 can comprise a monitoring software program 30 (MSP), a display 32, a user interface 34 and a database 36. The monitoring software program (MSP) 30 can be locally-installed on a computer, be located on a separate computer server and connected to a local computer through, for example, a private access connection (e.g. local area network), or be an application embedded in and accessible through a web server via the Internet. The computer can be a laptop computer, a desktop computer, a workstation, a handheld device, a server, a cluster of computers or other any suitable computing device. The MSP 30 can also be integrated into the processing unit 15. The database 36 can store historical status signals β for one or a multiple of subjects and can provide the historical data to the MSP 30.

Signals β representing the data may be combined with additional signals or data (e.g. generated by one or more additional sensors), filtered and relayed to the MSP 30 for generating content and displaying the content to a user. Additional sensors can include temperature sensors, light sensors, weight sensors, audio sensors, video, etc. The MSP 30 can generate content from the data (one or more parameters) as desired or required by the user and display the content associated with a particular subject through different screens on the display 32. The MSP 30 and display 32 can be connected to the user interface 34. The user interface 34 can include various user devices, such as a keyboard and mouse, a touch screen, stylus, microphone, etc.

The following description of displays in the monitoring program 100 and related interfaces illustrate exemplary content and screens generated by the MSP 30. The description and related figures reference use of the software program to generate content and display various parameters of subjects for use by various monitoring users. However, the references are merely exemplary and are not to limit the scope of embodiments of the invention. Those skilled in the art will realize that other embodiments may implement the MSP in the context of other areas.

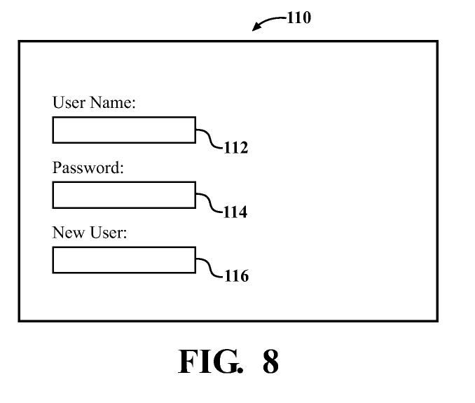

The MSP 30 described herein can generate and display a log-in screen 110 as seen in FIG. 8. The log-in screen can prompt a user to enter a previously registered user name 112 and password 114. In addition, a user can select a sign-in 116 option for new users to register to use the monitoring program. Registration can include the association of a subject and the subject's monitoring apparatus 10, such as a sensor ID, with the monitoring program 100. A user can be any person who is monitoring the subject or subjects for any reason. As non-limiting examples, the user could be the actual subject, medical facility personnel, home healthcare workers, family or friends caring for the subject, nursing homes, sleep clinics, trainers, prison discharge program operators, etc.

The MSP 30 can generate default content and use default displays that are preprogrammed for ease of use. The MSP 30 can also be modified by the user through the user interface 34 to generate and display content selected by the user. For example, in any of the display screens described herein, a default screen can include all of the options described, while a modified user-specific screen may display only the content in which the user is interested. Different users will be interested in different content. Examples of different uses will be described herein, illustrating how different users may be interested in different data and content.

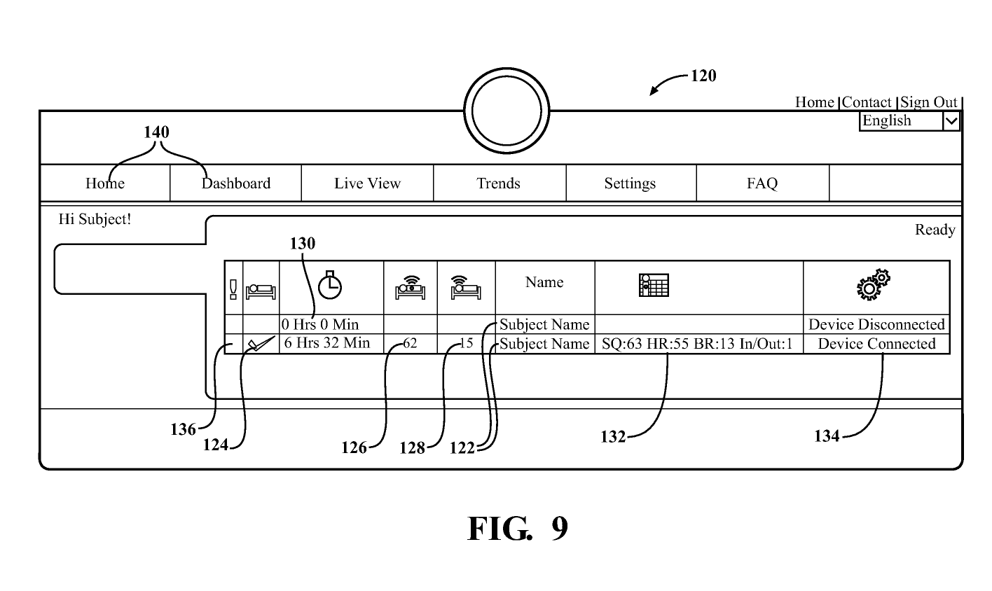

The MSP 30 can generate and display a home page 120 that provides an overview 122 of each monitoring system apparatus 10 available to the user to monitor, as shown in FIG. 9. For example, if a user is also the subject, the user may only see his self or herself listed on the home page 120. If the user is a parent, the user could see one or more children listed on the home page 120. If the user is a medical facility, the user may see any number of monitoring apparatus 10 listed by one or more of subject name, bed number and room number, for example. FIG. 9 is exemplary and illustrates a user monitoring two subjects each using a monitoring system apparatus 10 available to this user and identified by the subjects' names.

The home page 120 gives a brief overview 122 of each monitoring system 122 for which the user is registered to view. The home page 120 can provide information regarding one or more subjects with a quick review of the screen. The overview can indicate if the monitoring apparatus 10 is in active use 124, meaning that the subject is at rest against the bladder. As used in the figures, the symbol of a bed is used, with active use indicated by a subject on the bed and inactivity or non-use indicated by the bed without a subject. The bed is used as an example only. The system can be used in chairs to monitor a subject when the person is sitting if desired. If the monitoring apparatus 10 is active, the real time heart rate 126 and/or respiration 128 of the subject can be shown. The length of time 130 the subject has been being monitored can be included, which can indicate the amount of time the subject has been in bed. Any messages 132 entered by one of the users through the user interface 34 can also be generated and displayed. The home page 120 can also include whether the monitoring apparatus 10 is currently connected or disconnected 134, with disconnected systems indicating availability, for example, if the system is used in a hospital setting. The home page 120, as any of the pages, can be set up to display what is desired or required by a specific situation or user. The home page 120, as well as the other screens discussed herein, can have shortcuts 140 to quickly go to one of the other screens.

The home page 120, as well as any other page, can include an alarm indication 136. Alarms or alerts may be triggered by data that is outside a predetermined value range or meets pre-selected user trigger points. Simple alarms or alerts are audible and/or visible signals, such as horns, buzzers, sirens, lights, and the like. Alarms or alerts may be sent to pre-selected health care professionals (including paramedics, physicians, nurses, police, and the like), relatives and/or guardians, public health agencies, child services, etc., as determined by the user. Alarms, data messages and/or alerts may also be localized to particular places in a home, hospital, elderly, care facility, or infant care facility. Such signals may be transmitted by wired or wireless technology, such as cabling, WiFi, Zigbee, Bluetooth, etc., for contacting receiving devices such as cell phones or personal digital assistants (PDAs).

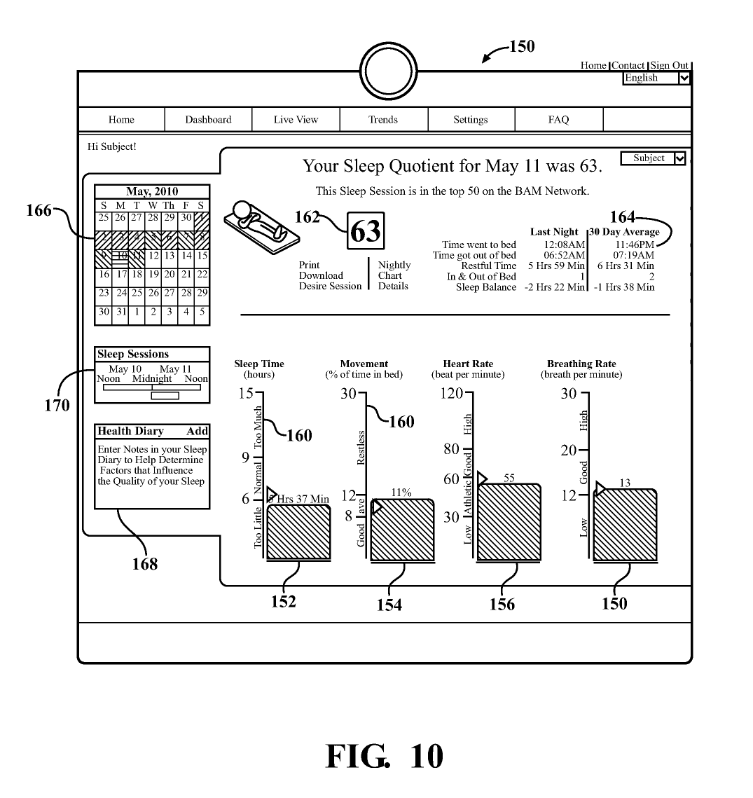

The MSP 30 can also generate content and display a dashboard 150, as shown in FIG. 10. The dashboard 150 is a detailed look at the data monitored by the user. If more than one subject or monitoring system 10 is listed in the overview 122 seen by the user, the user can select a subject or system 10 from the overview 122 on the home page 120, pulling up the dash board 150 for that particular subject. As illustrated in FIG. 10, the dashboard 150 can include information about a current or recent monitoring session, which can be a night's sleep, for example. Content such as the time the subject is asleep while being monitored, or sleep time 152 of the subject, can be generated and displayed, as well as amount of movement 154 of subject during the session, and average heart rate 156 and breathing rate 158 during the session. This content can be displayed in bar graph style as illustrated, with each data parameter shown against a scale 160 indicating whether or not the parameter is within a predetermined range. The predetermined range can be a default range, a normal or desirable value that is based on the subject's age, weight, sex, etc. and determined by a physician for example, or set based on governmental physical fitness standards. The predetermined range can also be set by the user through the user interface 34.

The dashboard 150 can also display the subject's sleep quotient 162 based on the subject's heart rate, respiratory rate, amount of time spend in REM sleep, total time in bed, and other considerations. A summary 164 can be provided of sleep patterns for the current session, as well as a historical average.

An interactive calendar 166 can be provided on the dashboard 150 that can provide an overview of a week or a month at one time. The user can select an earlier date to receive a historical overview of a particular data parameter, such as the sleep quotient. Selecting a specific date in the calendar 166 can bring the user to the dashboard 150 and other screens for the particular date selected. The days of the calendar 166 can be color coded to represent particular data as desired, such as sleep quotient, to quickly indicate to the user how frequently the subject's data is within the predetermined range. For example, dates in which the subject's sleep quotient is within range may be shaded green while dates in which the sleep quotient is out of the predetermined range may be in orange.

The dashboard 150 can also include a diary 168 in which the user can record incidents that may have bearing on why one or more parameters had a particular value that session. For example, a user may record environmental factors that influence sleep or vital signs, such as caffeine intake, alcohol intake, exercise performed, etc. The diary can provide the user information to analyze a particular pattern that develops. The dashboard 150 can also include a timeline 170 for the current monitoring session.

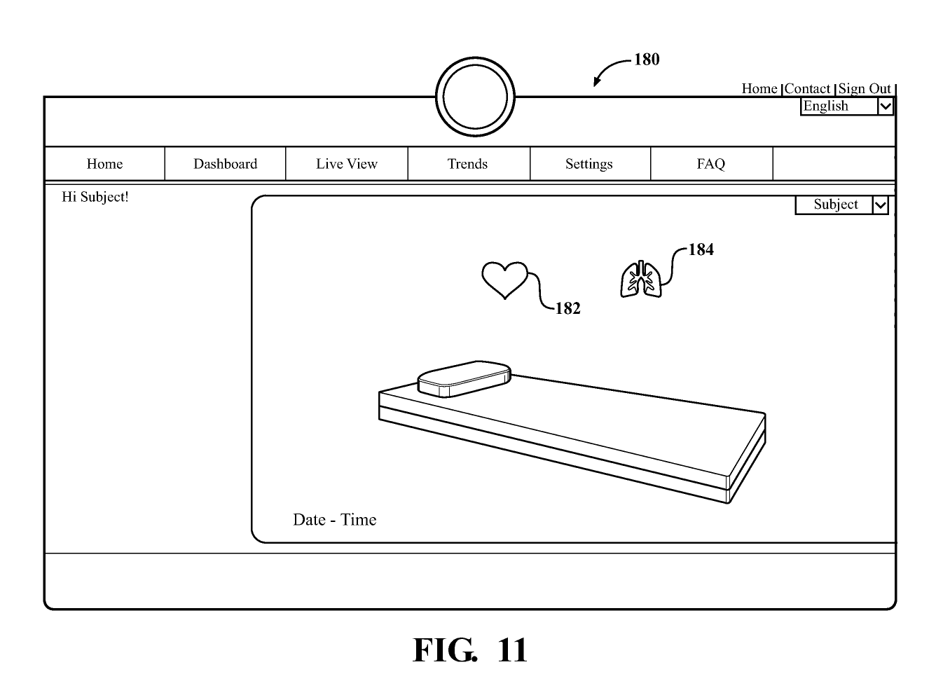

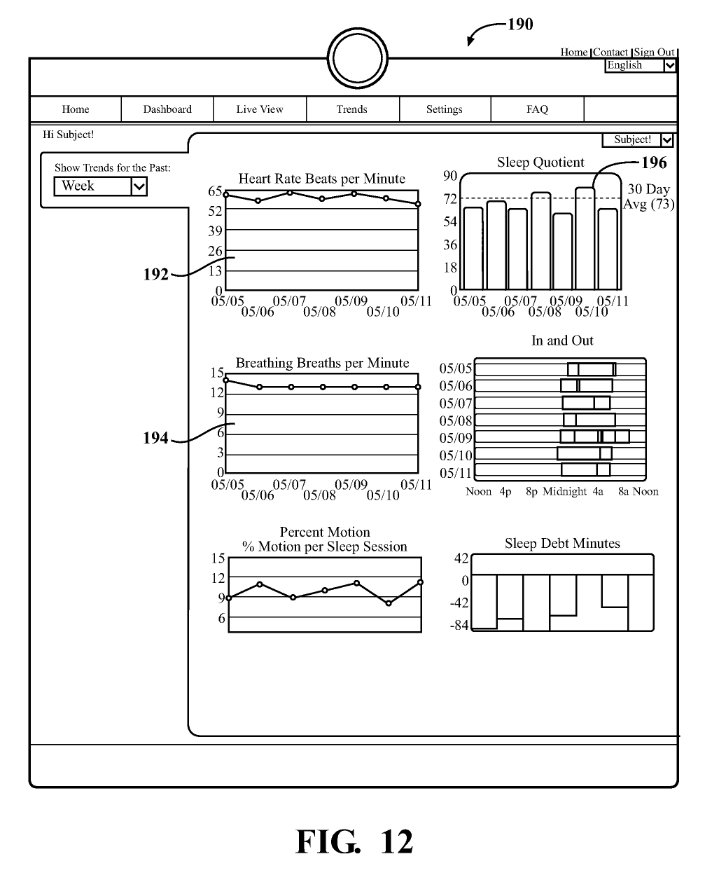

FIG. 11 illustrates a live view 180 screen generated and displayed by the MSP 30. The live view 180 can be a real time view of a particular subject's heart rate 182 and respiration 184, for example. The live view can also indicate whether the subject is currently asleep. The live view can also incorporate a live video stream of the subject. The date and time can also be displayed.

FIG. 12 illustrates a trends screen 190. As noted earlier, raw pressure values and data can be saved in the data base 36. The trends screen 190 provides graphical representations of the historical data from the data base 36 for one or more of the monitored parameters based on the default content generated or the content generated based on modifications of the user. For example, the trend screen 190 can display a line graph of the subject's hear rate 192 or respiration 194 for a predetermined period of time, such as a day, week or month. A bar graph can display a subject's average daily sleep quotient 196 for a selected period of time. Other trends can be displayed as desired or required by the user.

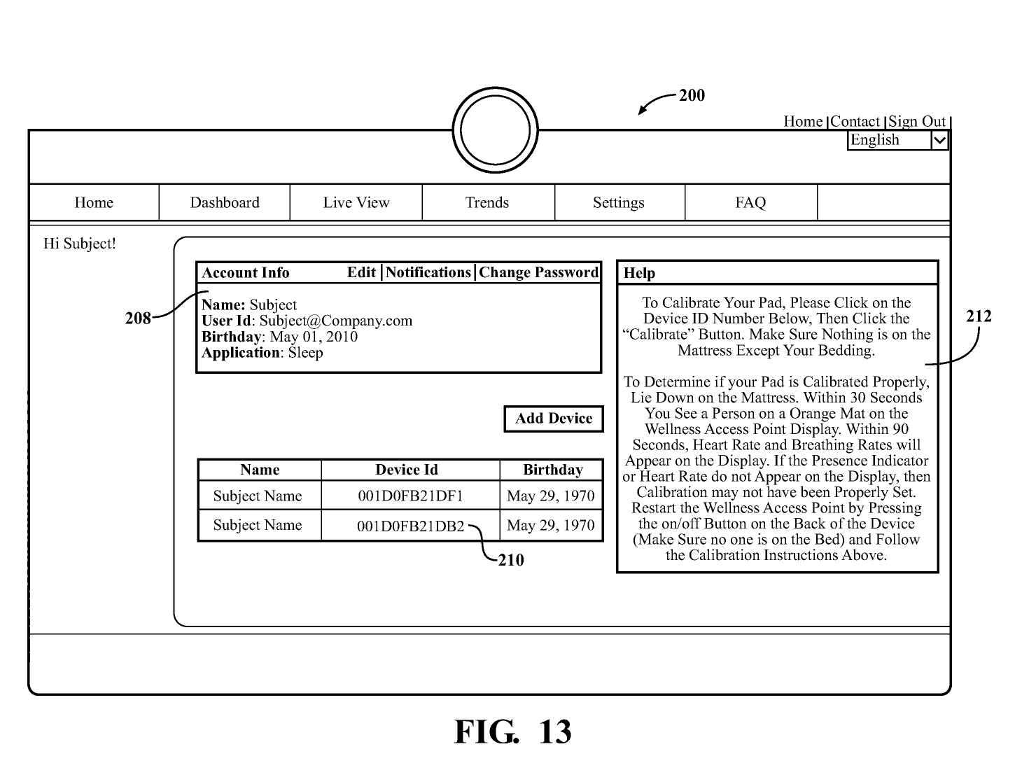

FIG. 13 illustrates a settings screen 200. The settings screen 200 can include account information 208, monitoring apparatus identification 210, such as serial number, calibration alerts or logs, etc. Particular screen setup information can also be entered in the settings screen 200, such as what data and trends the user would like displayed on the trend screen 190 or what information the user would like displayed on the dashboard 150. Predetermined periods can be set for displaying historical data, etc. For each subject, information such as weight, age, sex, etc. can be entered. The settings page can also include one or more help screens 212.

Any of the screens disclosed herein can include the capability to print the screen or the information displayed, download current and historical data, delete the information, etc. Furthermore, the MSP 30 is not limited to the particular displays herein. Other screen displays are contemplated that are within those skilled in the art and within the spirit and scope of the disclosure.

Following are non-limiting examples of how the monitoring system, including the apparatus 10 and monitoring program 100, can be used by various subjects and users. Other uses and users that are within the scope and spirit of the disclosure are contemplated.

As noted, the MSP 30 can be used to generate content and display for a plurality of system apparatus 10. A medical facility might use the monitoring system apparatus 10 in each bed, or each bed of a ward or floor, and use a single monitoring program 100 to monitor each of the beds with the MSP 30 generating and displaying content for each monitoring apparatus 10 on one or more displays 32. Each subject associated with a system apparatus 10 would be displayed on the home page 120, providing instant information to the user of what patients are in bed and what beds are available to new patients.

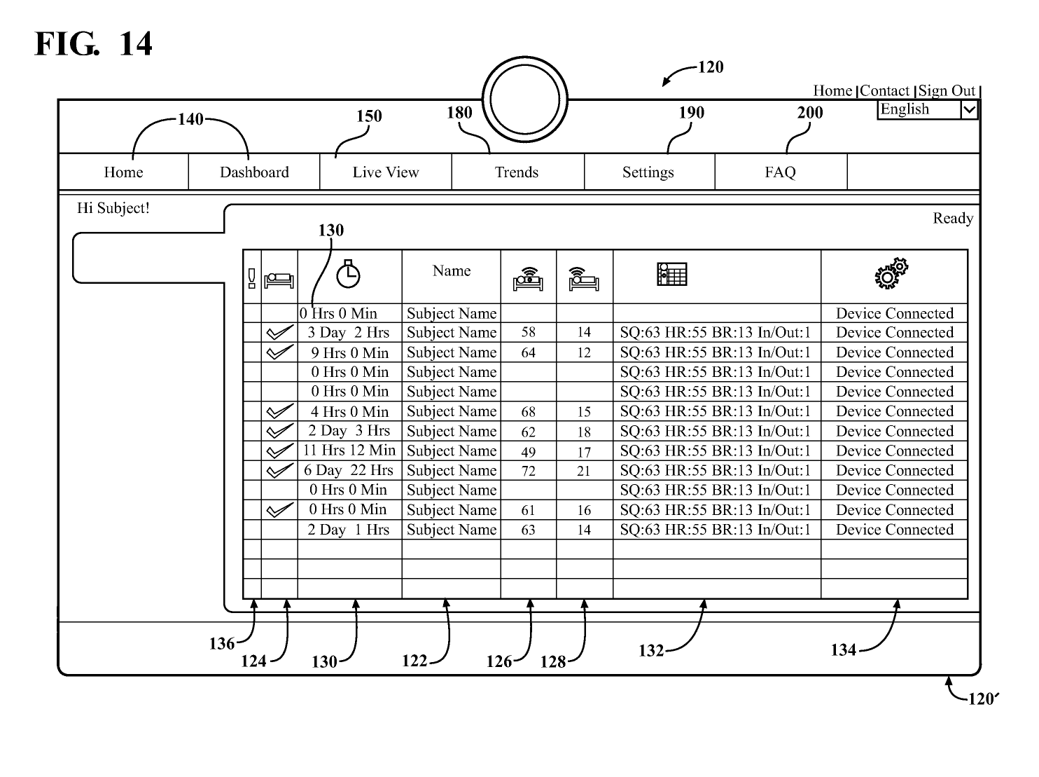

The monitoring system can be used to continually monitor the patients' vital signs. FIG. 14 is an example of a home page 120 that provides an instant view of the heart rate 126 and respiration 128 of a large number of patients. The data is real time data, as it can be updated at predetermined intervals such as every ten seconds. As mentioned earlier, an alarm indicator 136 can be included on the home page 120. The alarm indicator 136 can signal to a user when a patient is in distress, such as when the heart rate is too high or too low. The user can view the dashboard 150 of the patient by selecting the user from the overview 122 on the home page 120 to get a detailed view of the particular patient.

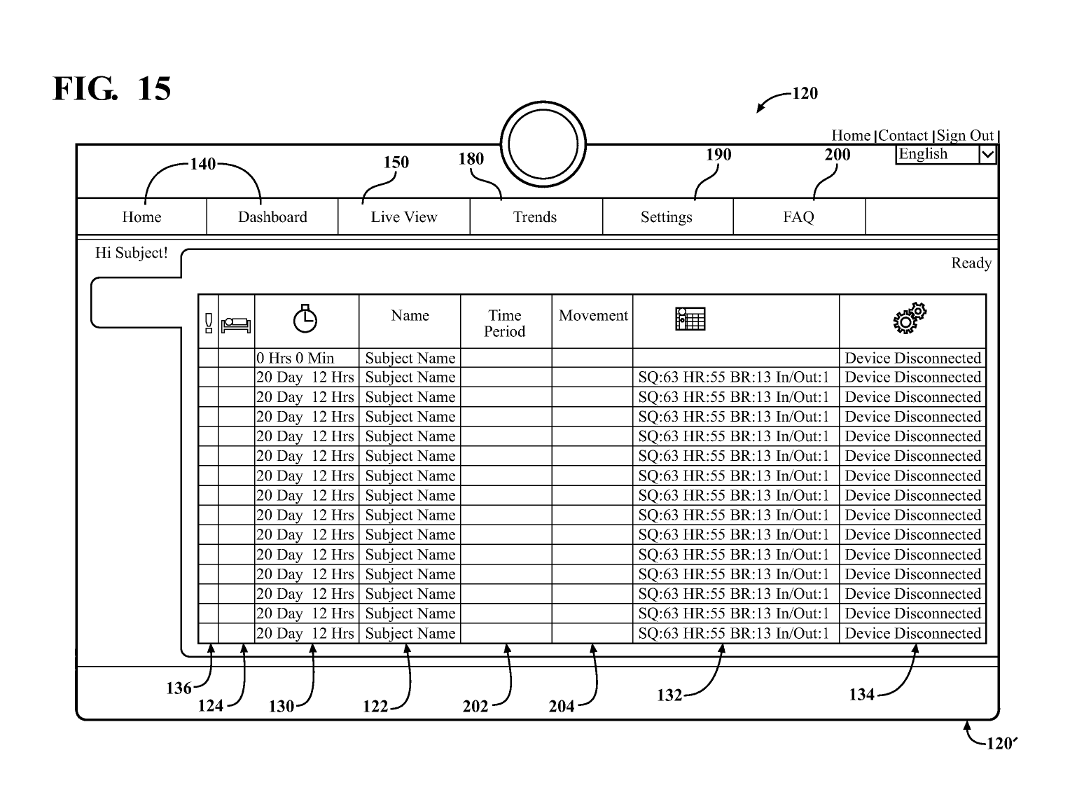

FIG. 15 is an example of a home page 120 that can be used to address the prevention of bed sores. Rather than having an instant view of heart rate and respiration, the home page 120 can provide an instant view of the length of time a patient is in bed 202 and a measurement of time between movements 204 sufficient to prevent bed sores. The alarm 136 can indicate when a patient must be turned if sufficient movement has not been detected. Messages 132 can be entered by staff to indicate, for example, when a patient was last moved by staff to indicate the frequency the staff had to assist. This can assist in tracking the amount of staff time and resources necessary to simply prevent bed sores, a typical and expensive problem in most medical facilities. The program can also monitor staff efficiency in responding to alerts, alarms or reminders for use by administration.

The monitoring system can be used by an individual subject at home, as the installation of the apparatus 10 is minimal and does not have any components that must be applied directly to the subject's body. The display 32 maybe implemented in any electronic device, such as a desktop or laptop computer, a handheld or portable computer-like device, MP3 and other electronic media players, cellular telephones, etc. The subject, also the user in this example, would register his apparatus 10 and himself on the log-in screen 110 of the monitoring program 100 to initiate communication between the apparatus 10 and the program 100. On the settings screen 200, the user would input through the user interface 34 the parameters he would like to monitor and how he would like the data displayed or use the default content and displays.

While the invention has been described in connection with what is presently considered to be the most practical example, it is to be understood that the invention is not to be limited to the disclosed example but, on the contrary, is intended to cover various modifications and equivalent arrangements included within the spirit and scope of the appended claims, which scope is to be accorded the broadest interpretation so as to encompass all such modifications and equivalent structures as is permitted under the law.

What is claimed is:

1. A device for monitoring a subject at rest comprising:
a sensing unit having a fluid-filled bladder configured to be placed under a substrate on which the subject lays and a sensor in fluid communication with the bladder, wherein the sensor is configured to sense pressure variations within the bladder generated by one or more of a heart beat, respiration and body weight of the subject and to generate signals indicative of the pressure variations;
a processor configured to receive the signals and to determine and generate output indicative of one or more of the subject's heart beat and respiration and presence on the substrate; and
an external device configured to display one or more of the output.

2. The device of claim 1, wherein the subject is a patient in a medical facility, the substrate is a mattress and the external device is operable to be monitored by personnel of the medical facility, the external device configured to display output for a plurality of patients each associated with a sensing unit.

3. The device of claim 2, wherein the external device comprises an alarm for alerting the personnel of output that is out of a predetermined range.

4. The device of claim 2, wherein the output generated by the processor further includes one or more of length of time on mattress, frequency of movement of patient on mattress and degree of movement by patient on the mattress, wherein frequency of movement is determined from number of changes in static pressure and degree of movement is determined from degree of change of static pressure and change of strength of one or more of heart beat and respiration.

5. The device of claim 4, wherein the external device is configured to generate an alarm based on the output, the alarm configured to generate an action by personnel to prevent pressure ulcers.

6. The device of claim 1, wherein the subject is a participant in a prison release program and the external device is operable to be monitored by personnel of the program, the external device configured to display output for a plurality of participants each associated with a sensing unit, the output including heart rate, respiration, and presence or absence in bed for each of the participants, wherein the output can be further analyzed to determine drug or alcohol abuse and compliance with curfews.

7. The device of claim 2 further comprising a database configured to store output over a period of time, wherein the stored output can be further analyzed to determine staffing requirements based on an amount of patient activity.

8. The device of claim 1 further comprising monitoring system software and a user interface, wherein the monitoring system software is configured to display the output in an arrangement based on user input through the user interface.

9. The device of claim 2, wherein the external device comprises a live view screen including a real-time status for each of the plurality of patients.

10. The device of claim 4, wherein the processor is further configured to calculate a sleep quotient based on the output for each of the plurality of patients.

11. A device for monitoring a subject comprising:
a sensing unit having a fluid-filled bladder configured to be placed on a substrate and a sensor in fluid communication with the bladder for generating signals in response to pressure variations within the bladder;
a processing unit configured to receive the signals from the sensor and to generate from the signals one or more of a heart rate, respiration rate and static pressure of the subject; and
means for providing to a user analyses based on one or more of the subject's heart rate, respiration rate and static pressure, wherein the analyses includes an indication of the subject in bed or out of bed.

12. The device of claim 11, wherein the processing unit is further configured to receive signals from a plurality of sensing units and wherein the user is personnel of a medical facility.

13. The device of claim 11, wherein the subject has dementia.

14. A method for monitoring a subject comprising:
sensing with a sensor incident pressure waves generated by the subject resting on a monitoring device, wherein the monitoring device comprises a fluid-filled bladder configured to be placed on a substrate with the sensor in fluid communication with the bladder;
generating signals indicative of the incident pressure waves and sending the signals to a processor;
discriminating heart beat, respiration and static pressure of the subject from analysis of the signals and determining an individual fingerprint based on heart beat, respiration and static pressure for the subject;
determining if the subject is in an assigned bed, not in the assigned bed, or in a different bed based on the presence or absence of the individual fingerprint; and
communicating the determination to an external device.

15. A device for preventing pressure ulcers in subjects comprising:
a sensing unit having a fluid-filled bladder configured to be placed on a substrate and a sensor in fluid communication with the bladder for generating signals in response to pressure variations within the bladder, wherein the pressure variations are generated from a heart rate, respiration rate and body weight of the subject;
a processing unit configured to receive the signals from the sensor and to determine from the signals a baseline heart rate, respiration rate and static pressure and to determine changes in strength of each of the heart rate, respiration rate and static pressure; and
means for providing to a user analyses based on the changes, wherein the analyses includes an indication of a need for a user action to prevent a pressure ulcer on the subject.

16. The device of claim 15, wherein the means for providing analysis comprises monitoring software program, a display and a user interface, the monitoring software program configured to receive data from the user through the user interface that the action has been completed.

17. A method of preventing pressure ulcers comprising:
sensing with a sensor incident pressure waves generated by the subject resting on a monitoring device, wherein the monitoring device comprises a fluid-filled bladder configured to be placed on a substrate with the sensor in fluid communication with the bladder;
generating signals indicative of the incident pressure waves and sending the signals to a processor;
determining a baseline heart rate, respiration and static pressure of the subject from the signals;
determining an amount of time the subject is resting on the monitoring device;
determining data indicating amount of movement and degree of movement of the subject while the subject is resting based on changes in static pressure of the bladder, changes in strength of heart beat and changes in strength of respiration;
determining if the subject requires an action by a user to prevent a pressure ulcer based on the data; and
communicating the required action to the user.

18. A portable biosensing device for use by a subject at home, the device comprising:
a bladder configured to be filled with a fluid and placed on a substrate;
a sensor in fluid communication with the bladder and configured to generate signals in response to pressure variations within the bladder, wherein the pressure variations are generated from a heart rate, respiration rate and body weight of the subject resting on the fluid-filled bladder;
a means for filling the bladder with fluid;
a processor configured to receive the signals from the sensor and determine the subject's heart rate and respiration rate; and
a database for storing the heart rate and respiration rate data.

19. The portable biosensing device of claim 18, wherein the subject is a discharged patient, the device further comprising communication means for communicating the stored data to a physician.

20. The portable biosensing device of claim 18, wherein the subject has an in-home care giver, the device further comprising a display with which the in-home care giver can monitor the subject.

21. The portable biosensing device of claim 20, wherein the processor is further configured to determine if a subject is in or out of bed.

22. The portable biosensing device of claim 18, wherein the subject is an infant, the device further comprising a display with which a parent of the infant can monitor the infant's heart rate and respiration.

23. The portable biosensing device of claim 18, wherein the processor is configured to calculate a sleep quotient for the subject from the subject's heart rate, respiration and changes in static pressure.
